(19) United States
(12) Patent Application Publication
(10) Pub. No.: US 2026/0083355 A1
(43) Pub. Date: Mar. 26, 2026

(54) BIOCHIP

(71) Applicant: VisEra Technologies Company Limited, Hsin-Chu City (TW)

(72) Inventors: Hsin-Yi HSIEH, Hsin-Chu City (TW); Chia-Chun CHANG, Hsin-Chu City (TW)

(21) Appl. No.: 18/891,535

(22) Filed: Sep. 20, 2024

(51) Int. Cl. A61B 5/145; A61B 5/1473; G02B 6/34

(52) U.S. Cl. CPC A61B 5/14514; A61B 5/1473; G02B 6/34

(57) ABSTRACT

A biochip is provided. The biochip includes a substrate and a waveguide core layer disposed over the substrate. The biochip also includes a waveguide core layer disposed over the substrate and a hydrogel. The waveguide core layer includes a grating coupler. The hydrogel is crosslinked via a hydrogel-crosslinking light that is coupled by the grating coupler.

### BACKGROUND OF THE INVENTION

### Field of the Invention

The present disclosure relates to a biochip, and, in particular, to a biochip that includes waveguide-assisted hydrogel crosslinking for biosample selective immobilization and multiplex detection.

### Description of the Related Art

Integrated sensing devices have recently become popular tools for biological analysis. When using such an application, a biological or biochemical sample may be placed on a biochip. The bio-reaction or interaction, such as DNA sequencing and immunofluorescence detection, may be reported through the excitation or emission spectrum or through the intensity of a fluorescent molecule. The fluorescent molecules may be excited by an excitation light with a shorter wavelength and generate an emission light with a longer wavelength toward the photoelectric conversion element (e.g., photodetector). The spectrum distribution and intensity of the fluorescence may be detected and measured by the photoelectric conversion element.

Although existing biochips have generally been adequate for their intended purposes, they have not been entirely satisfactory in all respects. Therefore, a novel biochip is still in demand.

### BRIEF SUMMARY OF THE INVENTION

In the embodiments of the present disclosure, a biochip that includes a waveguide core layer is provided. The waveguide core layer includes at least one grating coupler for coupling light to crosslink the hydrogel, so as to improve the selective modification and/or the multiplex screening capabilities of the biochip via thin hydrogel.

An embodiment of the present invention provides a biochip. The biochip includes a substrate and a waveguide core layer disposed over the substrate. The biochip also includes a waveguide core layer disposed over the substrate and a hydrogel. The waveguide core layer includes a grating coupler. The hydrogel is crosslinked via a hydrogel-crosslinking light that is coupled by the grating coupler.

In some embodiments, the substrate has a photoelectric conversion element, and the hydrogel corresponds to the photoelectric conversion element.

In some embodiments, the biochip further includes an upper cladding layer disposed on the waveguide core layer and including a nano-well that is disposed over the photoelectric conversion element. The upper cladding layer exposes the first grating coupler, and the hydrogel is disposed at the bottom of the nano-well.

In some embodiments, the thickness of the upper cladding layer is greater than 50 nm.

In some embodiments, when the thickness of the upper cladding layer is greater than 100 nm, the distance between the topmost of the hydrogel and the waveguide core layer is less than 100 nm.

In some embodiments, the substrate has multiple photoelectric conversion elements and the upper cladding layer includes multiple nano-wells that are disposed over the photoelectric conversion elements, and there are multiple hydrogels disposed at the bottoms of the nano-wells.

In some embodiments, the biochip further includes a self-assembled monolayer disposed on the upper cladding layer and between the upper cladding layer and the hydrogel.

In some embodiments, the first grating coupler is used for coupling a hydrogel-crosslinking light and a sensing light, and the wavelength of the hydrogel-crosslinking light is shorter than the wavelength of the sensing light.

In some embodiments, the first grating coupler is used for coupling a hydrogel-crosslinking light, and the waveguide core layer further includes a second grating coupler on the opposite side of the first grating coupler. The second grating coupler is used for coupling a sensing light, and the wavelength of the hydrogel-crosslinking light is shorter than the wavelength of the sensing light.

In some embodiments, the biochip further includes a lower cladding layer disposed between the substrate and the waveguide core layer.

In some embodiments, the waveguide core layer is formed as a channel waveguide. The channel waveguide includes at least one first grating coupler disposed over one side of the substrate, a second grating coupler disposed over another side of the substrate, and multiple lanes connecting the first grating coupler to the second grating coupler.

In some embodiments, there are multiple interleaved first grating couplers.

In some embodiments, the channel waveguide further includes a light-splitting component connecting the second grating coupler to the lanes.

In some embodiments, the first grating couplers are arranged in an array.

In some embodiments, the hydrogel is disposed on all of the lanes, and the hydrogel on different lanes has different functional molecules or concentrations.

In some embodiments, the first grating coupler is used for coupling light having the same or shorter wavelength than the light coupled by the second grating coupler.

In some embodiments, the hydrogel is disposed on one of the lanes to form a sensing arm, while the other of the lanes that is free of the hydrogel forms a reference arm.

In some embodiments, the hydrogel includes gelatin methacrylate, polyethylene glycol diacrylate, or hyaluronic acid.

In some embodiments, the hydrogel includes multiple functional molecules, and the functional molecules includes DNA primer, concanavalin A-dextran FRET complex, or antibodies.

In some embodiments, the biochip further includes a microneedle structure connecting the hydrogel to an external component.

### BRIEF DESCRIPTION OF THE DRAWINGS

The disclosure can be more fully understood from the following detailed description when read with the accompanying figures. It is worth noting that, in accordance with standard practice in the industry, various features are not drawn to scale. In fact, the dimensions of the various features may be arbitrarily increased or reduced for clarity of discussion.

FIG. 1A to FIG. 1J are three-dimensional schematic diagrams illustrating a method for manufacturing the biochip at various stages according to some embodiments of the present disclosure.

FIG. 2 is a cross-sectional view illustrating a portion of the biochip.

FIG. 3 illustrates a (enlarged) schematic diagram of the hydrogel and the functional molecules inside.

FIG. 4 is a three-dimensional schematic diagram illustrating the biochip according to some embodiments of the present disclosure.

FIG. 5 is a three-dimensional schematic diagram illustrating the biochip according to some other embodiments of the present disclosure.

FIG. 6A to FIG. 6O are three-dimensional schematic diagrams illustrating a method for manufacturing the biochip at various stages according to some embodiments of the present disclosure.

FIG. 7 is a three-dimensional schematic diagram illustrating the biochip according to some embodiments of the present disclosure.

FIG. 8A to FIG. 8E are three-dimensional schematic diagrams illustrating a method for manufacturing the biochip at various stages according to some embodiments of the present disclosure.

FIG. 9 is a three-dimensional schematic diagram illustrating the biochip according to some embodiments of the present disclosure.

FIG. 10 shows a schematic diagram of the effect of glucose on functional molecules in hydrogel of the biochip.

FIG. 11 is a glucose response spectrum.

FIG. 12 shows an application of the biochip on the skin of living organisms according to some embodiments of the present disclosure.

### DETAILED DESCRIPTION OF THE INVENTION

The following disclosure provides many different embodiments, or examples, for implementing different features of the subject matter provided. Specific examples of components and arrangements are described below to simplify the present disclosure. These are, of course, merely examples and are not intended to be limiting. For example, a first feature is formed on a second feature in the description that follows may include embodiments in which the first feature and second feature are formed in direct contact, and may also include embodiments in which additional features may be formed between the first feature and second feature, so that the first feature and second feature may not be in direct contact.

It should be understood that additional steps may be implemented before, during, or after the illustrated methods, and some steps might be replaced or omitted in other embodiments of the illustrated methods.

Furthermore, spatially relative terms, such as “beneath,” “below,” “lower,” “on,” “above,” “upper” and the like, may be used herein for ease of description to describe one element or feature's relationship to other elements or features as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

In the present disclosure, the terms “about,” “approximately” and “substantially” typically mean +/−20% of the stated value, more typically +/−10% of the stated value, more typically +/−5% of the stated value, more typically +/−3% of the stated value, more typically +/−2% of the stated value, more typically +/−1% of the stated value and even more typically +/−0.5% of the stated value. The stated value of the present disclosure is an approximate value. That is, when there is no specific description of the terms “about,” “approximately” and “substantially”, the stated value includes the meaning of “about,” “approximately” or “substantially”.

Unless otherwise defined, all terms (including technical and scientific terms) used herein have the same meaning as commonly understood by one of ordinary skill in the art to which this disclosure belongs. It should be understood that terms such as those defined in commonly used dictionaries should be interpreted as having a meaning that is consistent with their meaning in the context of the relevant art and will not be interpreted in an idealized or overly formal sense unless expressly so defined in the embodiments of the present disclosure.

The present disclosure may repeat reference numerals and/or letters in following embodiments. This repetition is for the purpose of simplicity and clarity and does not in itself dictate a relationship between the various embodiments and/or configurations discussed.

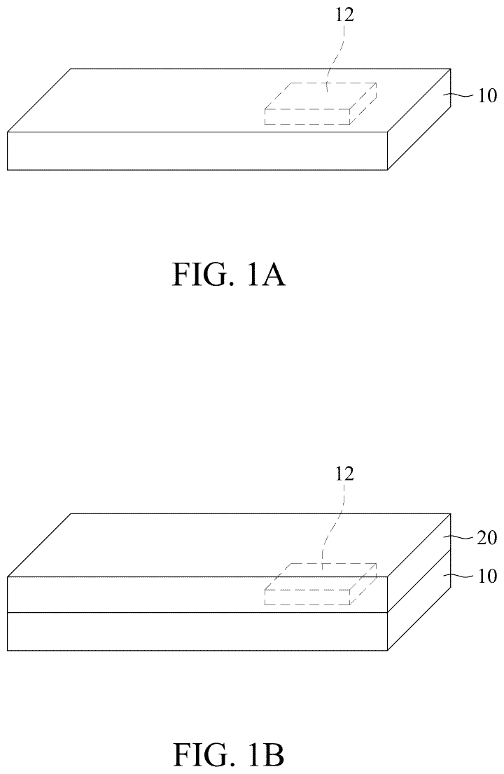

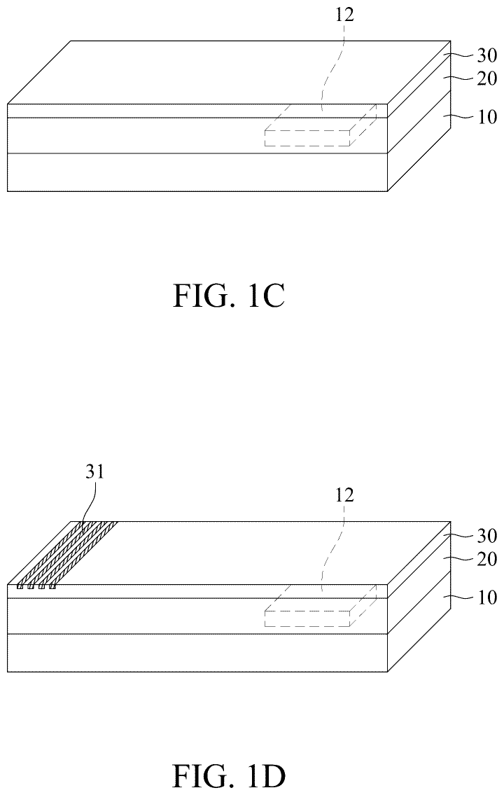

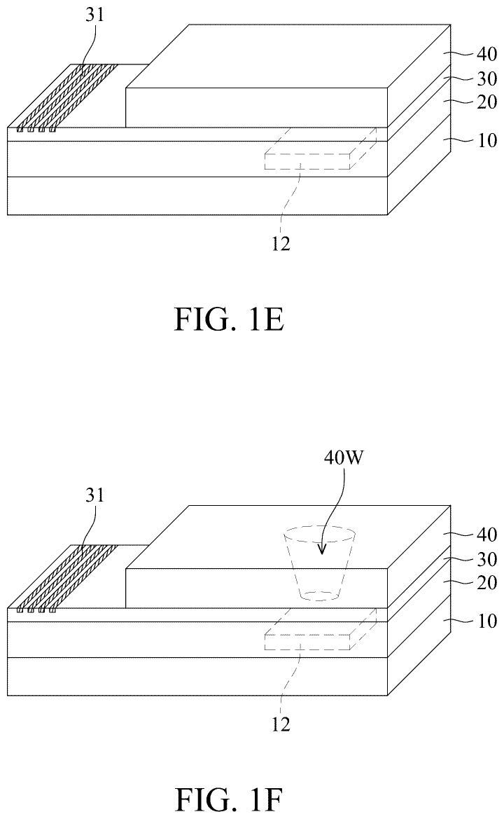

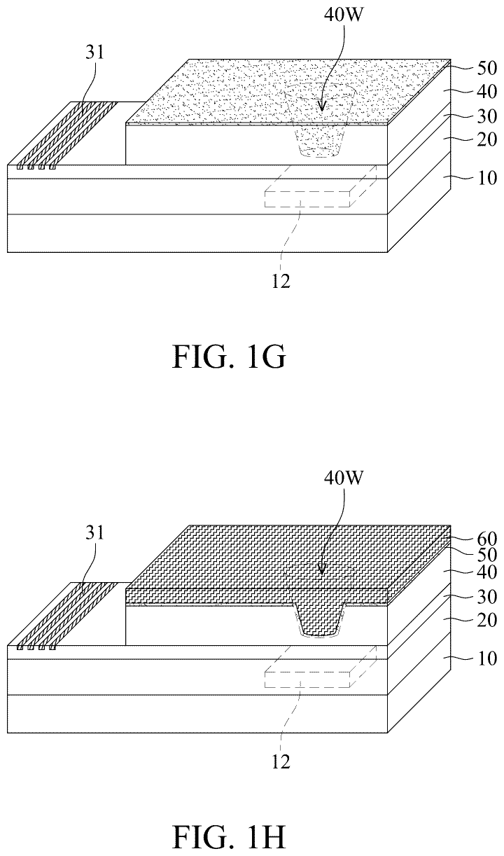

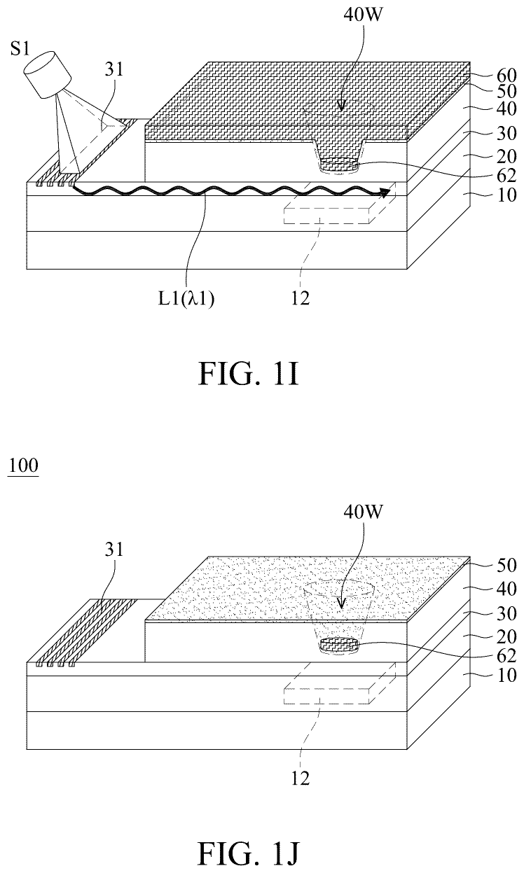

FIG. 1A to FIG. 1J are three-dimensional schematic diagrams illustrating a method for manufacturing the biochip 100 at various stages according to some embodiments of the present disclosure. It should be noted that some components have been omitted in FIG. 1A to FIG. 1J for the sake of brevity.

Referring to FIG. 1A, in some embodiments, a substrate 10 that has a photoelectric conversion element 12 is provided. In some embodiments, substrate 10 is a glass substrate or a semiconductor substrate (e.g., CMOS substrate), and the photoelectric conversion element 12 is a photodiode. For example, the substrate 10 may include a flexible material, such as polyethylene terephthalate (PET), polysulfone (PES), polyimide (PI), polycarbonate (PC), polymethylmethacrylate (PMMA), silicone, epoxy, the like, or a combination thereof. The substrate 10 may also include a rigid material, such as a glass, a quartz, or a sapphire.

The substrate 10 may be transparent or semi-transparent. More specifically, in the examples where the substrate 10 is transparent, the material of the substrate 10 may have a light transmittance to light with a wavelength in a range from 400 nm to 750 nm greater than about 85%, or greater than about 92%. In the examples where the substrate 10 is semi-transparent, the material of the substrate 10 may have a light transmittance to light with a wavelength in a range from 400 nm to 750 nm greater than about 25% and less than about 85%, but the present disclosure is not limited thereto.

Referring to FIG. 1B, in some embodiments, a lower cladding layer 20 is formed on the substrate 10. For example, the lower cladding layer 20 may include a transparent dielectric material that has a low refractive index in a range from about 1.0 to about 1.99, such as silicon dioxide (SiO₂), but the present disclosure is not limited thereto. The lower cladding layer 20 may be formed by a deposition process. The deposition process is, for example, chemical vapor deposition (CVD), plasma enhanced chemical vapor deposition (PECVD), atomic layer deposition (ALD), any other similar process, or a combination thereof, but the present disclosure is not limited thereto. It should be noted that the lower cladding layer 20 may be omitted in the embodiment where the substrate 10 is a glass substrate.

Referring to FIG. 1C, in some embodiments, a waveguide core layer 30 is formed over the substrate 10. In other words, the lower cladding layer 20 is disposed between the substrate 10 and the waveguide core layer 30. For example, the waveguide core layer 30 may include silicon nitride (SiN), tantalum oxide (TaO), titanium(II) oxide (TiO), aluminum(II) oxide (AlO), any other similar material, or a combination thereof, but the present disclosure is not limited thereto. The waveguide core layer 30 may be formed by a deposition process. Examples of the deposition process is described above and will not be repeated here.

Referring to FIG. 1D, in some embodiments, a grating coupler 31 is formed on one side of the waveguide core layer 30. The grating coupler 31 may be formed by a photolithography process and/or an etching process. For example, the photolithography process may include photoresist coating (e.g., spin coating), soft baking, mask aligning, exposure, post-exposure baking (PEB), developing, rinsing, drying (for example, hard baking), any other suitable process, or a combination thereof, but the present disclosure is not limited thereto.

Referring to FIG. 1E, in some embodiments, an upper cladding layer 40 is formed on the waveguide core layer 30. As shown in FIG. 1E, the upper cladding layer 40 exposes the grating coupler 31. The upper cladding layer 40 may include the same or similar material to the lower cladding layer 20 and be formed by the same or similar process, which will not be repeated here.

Referring to FIG. 1F, in some embodiments, the upper cladding layer 40 is patterned to form a nano-well (a vial) 40W. As shown in FIG. 1F, the nano-well 40W is disposed over (or corresponds to) the photoelectric conversion element 12. In some embodiments, the nano-well 40W is close to but does not penetrate the upper cladding layer 40. That is, there is a gap between the bottommost of the nano-well 40 and the waveguide core layer 30.

The nano-well 40W may be formed by a patterning process. The patterning process may include, for example, forming a mask layer (not illustrated) on the upper cladding layer 40, then etching the portion of the upper cladding layer 40 that is not covered by the mask layer, but the present disclosure is not limited thereto.

The mask layer may include a photoresist, such as a positive photoresist or a negative photoresist. For example, the mask layer may include a metal, a metal oxide, a metal nitride (e.g., Ti, TiO₂, TiN, Al, Al₂O₃, AlN, Cr, or Nb,) or a dielectric material (e.g., silicon oxide (SiO₂), silicon nitride (SiN), silicon oxynitride (SiON), silicon carbide (SiC), silicon carbonitride (SiCN)), or a combination thereof. The mask layer may be a single layer or a multilayer structure.

Referring to FIG. 1G, in some embodiments, a self-assembled monolayer (SAM) 50 is formed on the upper cladding layer 40. In more detail, the self-assembled monolayer 50 is formed on the top surface of the upper cladding layer 40 and in the nano-well 40W (i.e., on the bottom and sidewall of the upper cladding layer 40). For example, the self-assembled monolayer 50 may include silicon-hydrogen compounds (silanes), but the present disclosure is not limited thereto. The self-assembled monolayer 50 may be formed by a deposition process, such as a (spin-on or vapor) coating process.

Referring to FIG. 1H, in some embodiments, a hydrogel prepolymer 60 is formed on the self-assembled monolayer 50. In this embodiment, the hydrogel prepolymer 60 is formed in the nano-well 40W of the upper cladding layer 40. Moreover, the hydrogel prepolymer 60 may fully fill the nano-well 40W of the upper cladding layer 40, but the present disclosure is not limited thereto. Here, the hydrogel prepolymer 60 is non-crosslinked and may be formed by a deposition process.

Referring to FIG. 1I, in some embodiments, a hydrogel-crosslinking light L1 is coupled by the grating coupler 31 to form a (crosslinked) hydrogel 62 that corresponds to the photoelectric conversion element 12. In more detail, a light source S1 emits light to the grating coupler 31 of the waveguide core layer 30, and a hydrogel-crosslinking light L1 having a wavelength of λ1 is formed and passes through the waveguide core layer 30. Since the nano-well 40W is close to the waveguide core layer 30, a portion of hydrogel prepolymer 60 in the bottom of the nano-well 40W is crosslinked via the hydrogel-crosslinking light L1 that is coupled by the grating coupler 31 to form the hydrogel 62. That is, in some embodiments, the hydrogel 62 is disposed at the bottom of the nano-well 40W.

Here, the uncrosslinked hydrogel prepolymer 60 may have a photo-initiator that includes a UV light-based initiator (e.g., Irgacure2959) or a visible light-based initiator (e.g., Eosin Y or Lithium phenyl-2,4,6-trimethylbenzoylphosphinate (LAP)), and the hydrogel-crosslinking light L1 may have the wavelength in the light sensitive region of the photo-initiator. In some embodiments, the hydrogel 62 includes gelatin methacrylate (GelMA), polyethylene glycol diacrylate (PEGDA), or hyaluronic acid (HA).

Referring to FIG. 1J, in some embodiments, another portion of hydrogel prepolymer 60 that is not crosslinked via the hydrogel-crosslinking light L1 is removed to form the biochip 100. In other words, the uncrosslinked hydrogel prepolymer 60 is washed out, so that the self-assembled monolayer 50 and the hydrogel 62 are exposed.

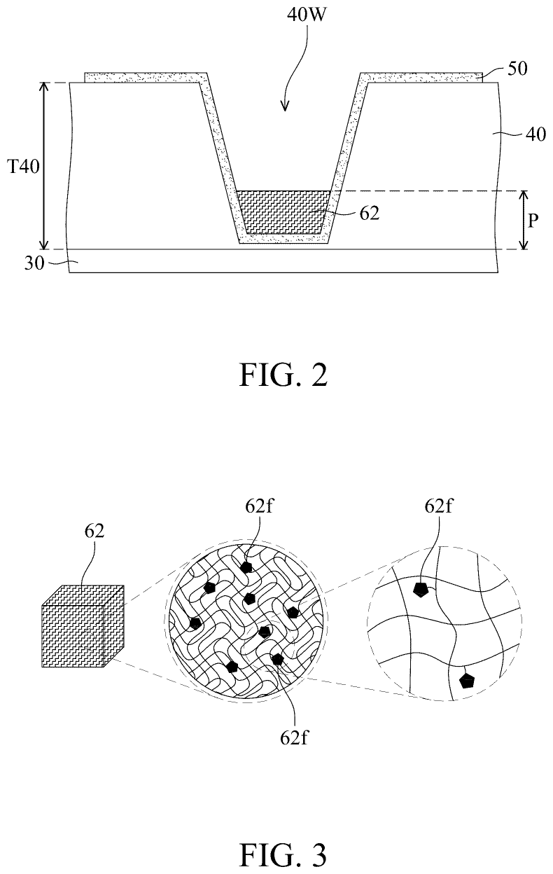

FIG. 2 is a cross-sectional view illustrating a portion of the biochip 100. In more detail, FIG. 2 shows the waveguide core layer 30, the upper cladding layer 40, the self-assembled monolayer 50, and the hydrogel 62. As shown in FIG. 1J and FIG. 2, in this embodiment, the self-assembled monolayer 50 is disposed on the upper cladding layer 40 and between the upper cladding layer 40 and the hydrogel 62. In more detail, the self-assembled monolayer 50 is disposed on the top surface of the upper cladding layer 40 and on the bottom and sidewall of the nano-well 40W.

As shown in FIG. 2, in some embodiments, the thickness T40 of the upper cladding layer 40 is greater than about 50 nm, which may isolate the hydrogel prepolymer 60 from being crosslinked at the evanescent wave region (i.e., the pathway of the hydrogel-crosslinking light L1) of the waveguide core layer 30. As shown in FIG. 2, in some embodiments, when the thickness T40 of the upper cladding layer 40 is greater than about 100 nm, the distance P between the topmost of the hydrogel 62 and the waveguide core layer 30 is less than about 100 nm, which may be less than the penetration depth of wavelength of λ1 (e.g., about 30-50 nm), so that the portion of hydrogel prepolymer 60 in the bottom of the nano-well 40W may be crosslinked via the hydrogel-crosslinking light L1. Moreover, the top surface of the hydrogel 62 may be an area for binding a biosample.

FIG. 3 illustrates a (enlarged) schematic diagram of the hydrogel 62 and the functional molecules 62f inside. As shown in FIG. 3, in some embodiments, the hydrogel 62 includes multiple functional molecules 62f, which may bind analytes with fluorescent tags. Moreover, in some embodiments, the functional molecules 62f includes DNA primer, concanavalin A (Con A)-dextran FRET complex, or antibodies. For example, the DNA primer may immobilize DNA fragment for DNA hybridization or DNA sequencing, the Concanavalin A (Con A)-Dextran FRET complex may detect glucose molecule, and the antibodies may bind a specific antigen or analyte from a neighboring cell for screening cell phenotype.

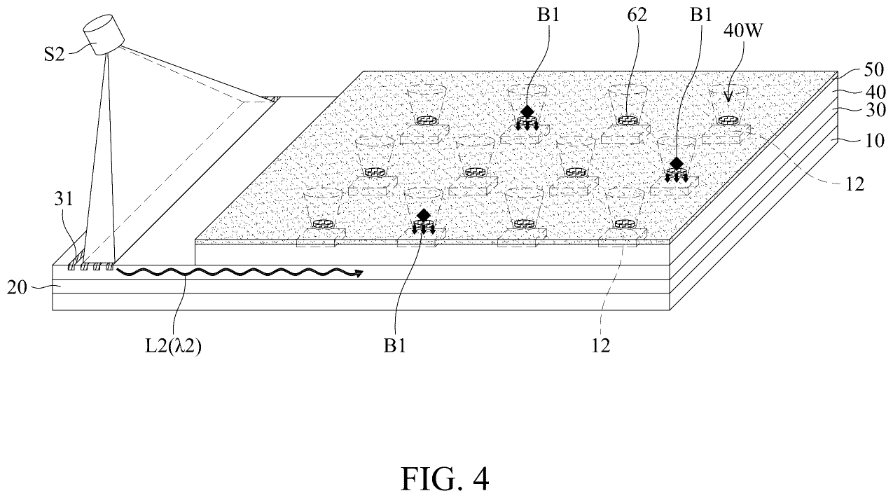

FIG. 4 is a three-dimensional schematic diagram illustrating the biochip 100 according to some embodiments of the present disclosure. It should be noted that some components of the biochip 100 have been omitted in FIG. 4 for the sake of brevity. As shown in FIG. 4, in some embodiments, the biochip 100 includes a substrate 10 and a waveguide core layer 30 disposed over the substrate 10. The substrate 10 has a photoelectric conversion element 12. The biochip 100 also includes a waveguide core layer 30 disposed over the substrate 10 and a hydrogel 62 corresponding to the photoelectric conversion element 12. The waveguide core layer 30 includes a grating coupler 31. The hydrogel 62 is crosslinked via a hydrogel-crosslinking light (L1 shown in FIG. 1I) that is coupled by the grating coupler 31.

In this embodiment, the biochip 100 further includes a lower cladding layer 20 and an upper cladding layer 40. The lower cladding layer 20 is disposed between the substrate 10 and the waveguide core layer 30, and the upper cladding layer 40 is disposed on the waveguide core layer 30. The upper cladding layer 40 includes a nano-well 40W that is disposed over the photoelectric conversion element 12. The upper cladding layer 40 exposes the grating coupler 31, and the hydrogel 62 is disposed at the bottom of the nano-well 40W. Moreover, the biochip 100 includes a self-assembled monolayer 50 disposed on the upper cladding layer 40 and between the upper cladding layer 40 and the hydrogel 62.

As shown in FIG. 4, in some embodiments, the substrate 10 has multiple photoelectric conversion elements 12 and the upper cladding layer includes multiple nano-wells 40W that are disposed over the photoelectric conversion elements 12, and there are multiple hydrogels 62 disposed at the bottoms of the nano-wells 40W.

In this embodiment, the grating coupler 31 is used for coupling the hydrogel-crosslinking light L1 (see FIG. 1I) and a sensing light L2, and the wavelength λ1 of the hydrogel-crosslinking light L1 is shorter than the wavelength λ2 of the sensing light L2. As shown in FIG. 4, a light source S2 emits light to the grating coupler 31 of the waveguide core layer 30, and a sensing light L2 having a wavelength of λ2 is formed and passes through the waveguide core layer 30. the sensing light L2 having the wavelength of λ2 may excite fluorescence molecules to emit fluorescent light that can be collected by the photoelectric conversion elements 12 (e.g., photodiodes) under the nano-wells 40W. In other words, the hydrogel-crosslinking light L1 (having the wavelength of λ1) and the sensing light L2 (having the wavelength of λ2) may be coupled into the waveguide core layer 30 by the same grating coupler 31. That is, the grating coupler 31 may perform optical coupling for λ1 and λ2 at the same time, and the optical coupling angles may be different (λ1≤λ2) However, the present disclosure is not limited thereto.

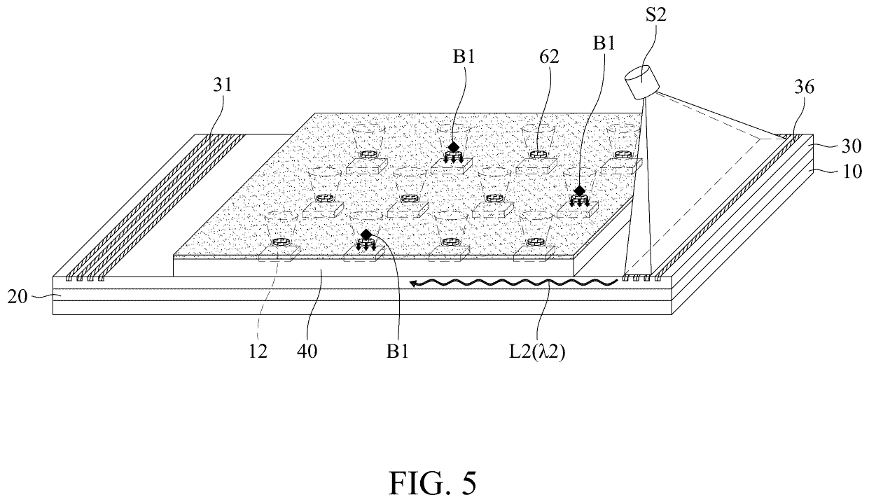

FIG. 5 is a three-dimensional schematic diagram illustrating the biochip 100′ according to some other embodiments of the present disclosure. Similarly, some components of the biochip 100′ have been omitted in FIG. 5 for the sake of brevity. As shown in FIG. 5, in some embodiments, the biochip 100 includes the grating coupler 31 is used for coupling a hydrogel-crosslinking light L1 (see FIG. 1I) and the waveguide core layer 30 further includes a grating coupler 36 on the opposite side of the grating coupler 31. The grating coupler 36 is used for coupling a sensing light L2, and the wavelength λ1 of the hydrogel-crosslinking light L1 is shorter than the wavelength λ2 of the sensing light L2.

In other words, in this embodiment, the hydrogel-crosslinking light L1 and the sensing light L2 may be designed to be coupled and guided by different grating couplers (e.g., grating coupler 31 and grating coupler 36) and set to the same grating depth. The period and fill factor of different grating coupler designs may be optimized for different light sources (e.g., light source S1 and light source S2) to match the required coupling angle and incident light wavelength.

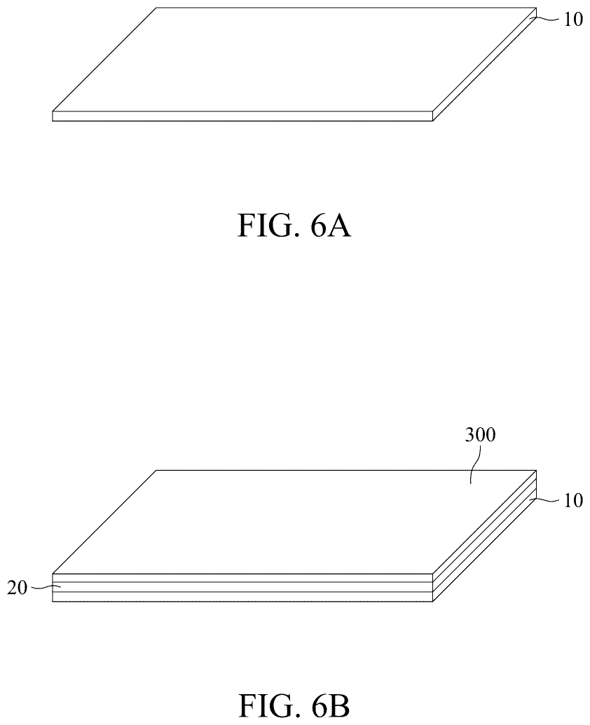

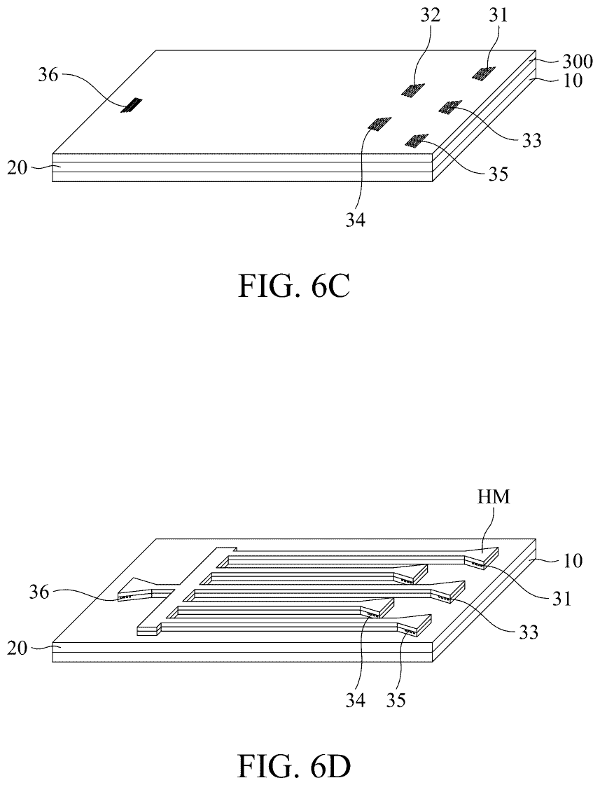

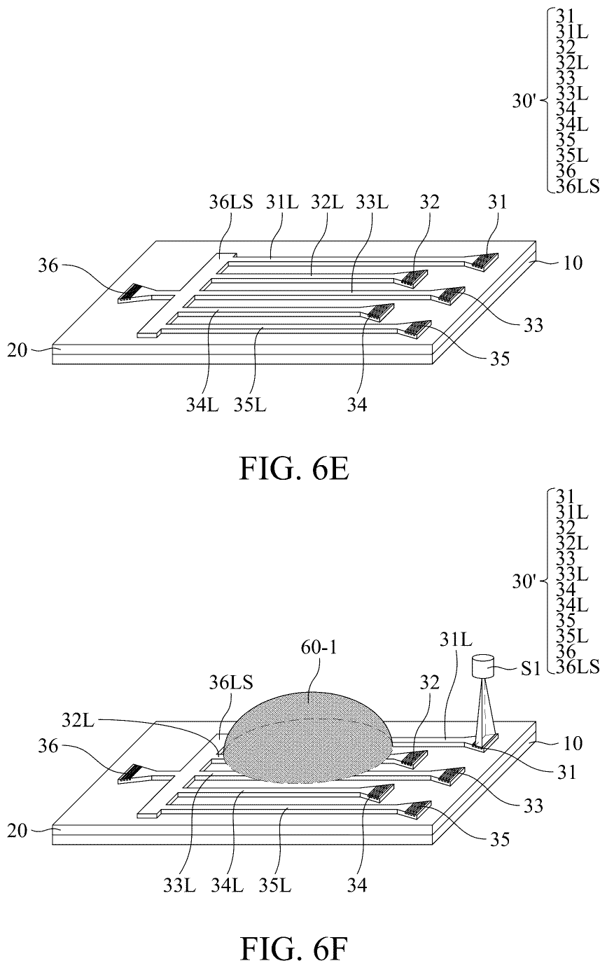

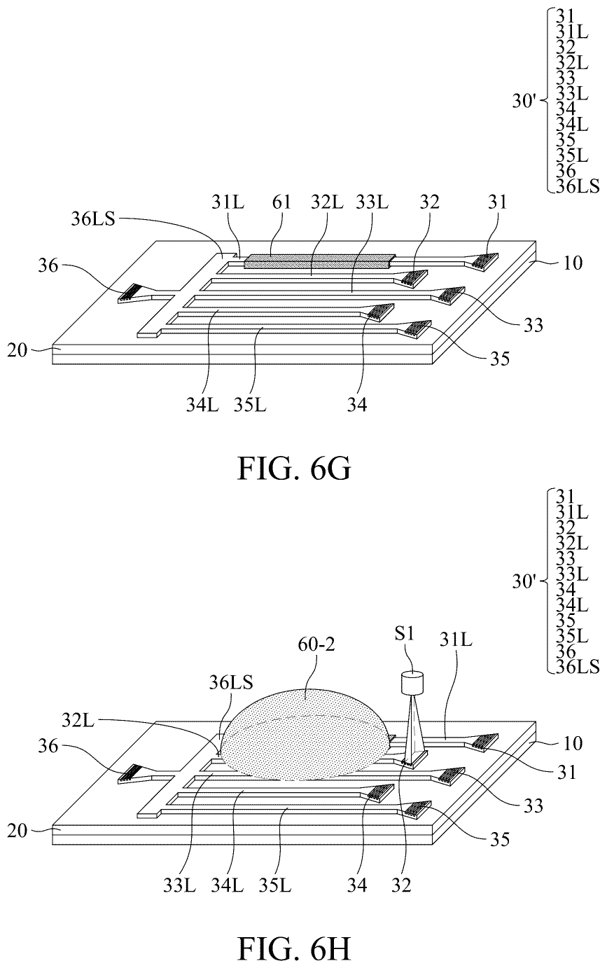

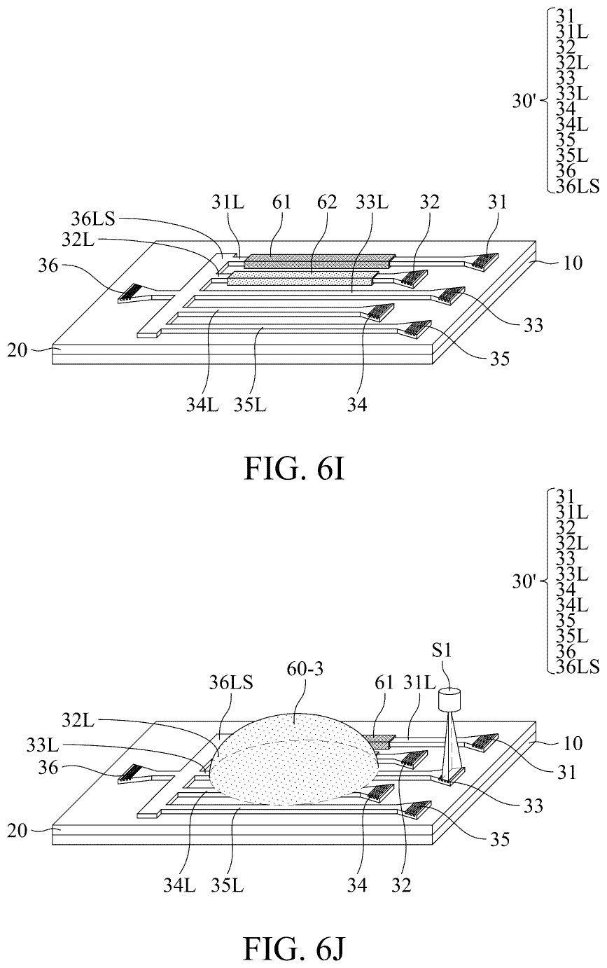

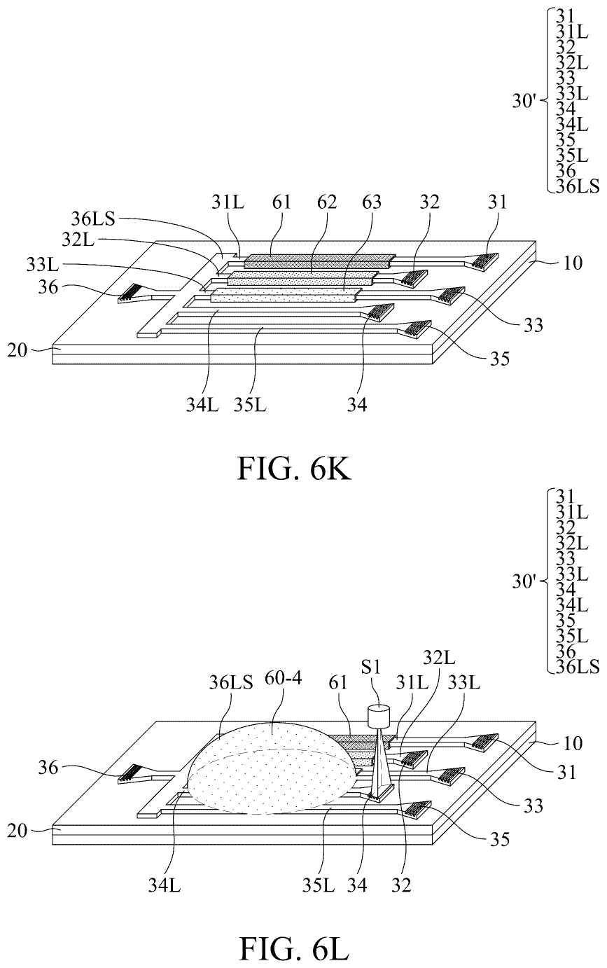

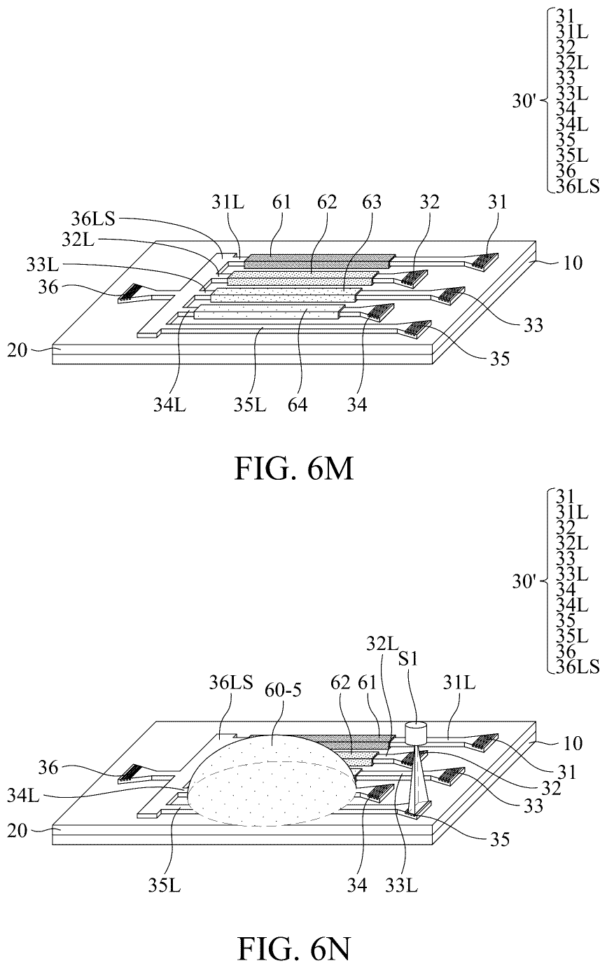

FIG. 6A to FIG. 6O are three-dimensional schematic diagrams illustrating a method for manufacturing the biochip 102 at various stages according to some embodiments of the present disclosure. It should be noted that some components have been omitted in FIG. 6A to FIG. 6O for the sake of brevity.

Referring to FIG. 6A, in some embodiments, a substrate 10 is provided. It should be noted that the substrate 10 may also have multiple photoelectric conversion elements (not shown in FIG. 6A to FIG. 6O). Referring to FIG. 6B, in some embodiments, a lower cladding layer 20 and a waveguide material layer 300 are sequentially formed on the substrate 10. Similarly, the lower cladding layer 20 may be omitted in the embodiment where the substrate 10 is a glass substrate.

Referring to FIG. 6C, in some embodiments, grating couplers 31, 32, 33, 34, and 35 are formed on one side of the waveguide material layer 300 (which are disposed over one side of the substrate 10), while a grating coupler 36 is formed on another side of the waveguide material layer 300 (which is disposed over another side of the substrate 10). The grating couplers 31, 32, 33, 34, 35, and the grating coupler 36 may be formed by a photolithography process and/or an etching process.

Referring to FIG. 6D and FIG. 6E, in some embodiments, a waveguide material layer 300 is patterned to form a channel waveguide 30′. In more detail, an interdigitated mask layer HM may be formed on the waveguide material layer 300, then a portion of the waveguide material layer 300 not covered by the mask layer HM is removed to form the channel waveguide 30′. In other words, the waveguide material layer 300 not covered by the mask layer HM may be etched, thereby retaining the channel waveguide 30′.

As shown in FIG. 6E, in some embodiments, the channel waveguide 30′ includes the grating coupler 31, 32, 33, 34, and 35 disposed over one side (e.g., right side in FIG. 6E) of the substrate 10, the grating coupler 36 disposed over another side (e.g., left side in FIG. 6E) of the substrate 10, and multiple lanes 31L, 32L, 33L, 34L, and 35L that respectively connect the grating coupler 31, 32, 33, 34, and 35 to the grating coupler 36. In this embodiment, the grating couplers 31, 32, 33, 34, and 35 are interleaved grating couplers, so that the arrangement of grating coupler 31, 32, 33, 34, and 35 may be denser. As shown in FIG. 6E, in some embodiments, the grating couplers 31, 32, 33, 34, and 35 are arranged in an array.

It should be noted that the number of grating coupler 31, 32, 33, 34, and 35 (which are disposed over the right side of the substrate 10 in FIG. 6E) is not limited to five, which may be adjusted according to actual needs. Moreover, in some embodiments, the channel waveguide 30′ further includes a light-splitting component 36LS that connects the grating coupler 36 to the lanes 31L, 32L, 33L, 34L, and 35L.

Referring to FIG. 6F, in some embodiments, a hydrogel prepolymer 60-1 is formed (dropped) on the channel waveguide 30′ and at least in contact with the lane 31L. Then, a light source S1 emits a hydrogel-crosslinking light (not labeled in FIG. 6F) having a wavelength of λ1 into the grating coupler 31, and the hydrogel-crosslinking light passes through the lane 31L. Referring to FIG. 6G, in some embodiments, a (crosslinked) hydrogel 61 is formed on the lane 31L. Since the hydrogel-crosslinking light merely passes through the lane 31L, a portion of the hydrogel prepolymer 60-1 on the lane 31L may be is crosslinked via the hydrogel-crosslinking light, while other portions of the hydrogel prepolymer 60-1 that are not crosslinked may be removed (e.g., washed out).

Referring to FIG. 6H, in some embodiments, a hydrogel prepolymer 60-2 is formed (dropped) on the channel waveguide 30′ and at least in contact with the lane 32L. Then, a light source S1 emits a hydrogel-crosslinking light (not labeled in FIG. 6H) having a wavelength of λ1 to the grating coupler 32, and the hydrogel-crosslinking light passes through the lane 32L. Referring to FIG. 6I, in some embodiments, a (crosslinked) hydrogel 62 is formed on the lane 32L. Since the hydrogel-crosslinking light merely passes through the lane 32L, a portion of the hydrogel prepolymer 60-2 on the lane 32L may be is crosslinked via the hydrogel-crosslinking light, while other portions of the hydrogel prepolymer 60-2 that are not crosslinked may be removed (e.g., washed out).

Referring to FIG. 6J, in some embodiments, a hydrogel prepolymer 60-3 is formed (dropped) on the channel waveguide 30′ and at least in contact with the lane 33L. Then, a light source S1 emits a hydrogel-crosslinking light (not labeled in FIG. 6J) having a wavelength of λ1 to the grating coupler 33, and the hydrogel-crosslinking light passes through the lane 33L. Referring to FIG. 6K, in some embodiments, a (crosslinked) hydrogel 63 is formed on the lane 33L. Since the hydrogel-crosslinking light merely passes through the lane 33L, a portion of the hydrogel prepolymer 60-3 on the lane 33L may be is crosslinked via the hydrogel-crosslinking light, while other portions of the hydrogel prepolymer 60-3 that are not crosslinked may be removed (e.g., washed out).

Referring to FIG. 6L, in some embodiments, a hydrogel prepolymer 60-4 is formed (dropped) on the channel waveguide 30′ and at least in contact with the lane 34L. Then, a light source S1 emits a hydrogel-crosslinking light (not labeled in FIG. 6L) having a wavelength of λ1 to the grating coupler 34, and the hydrogel-crosslinking light passes through the lane 34L. Referring to FIG. 6M, in some embodiments, a (crosslinked) hydrogel 64 is formed on the lane 34L. Since the hydrogel-crosslinking light merely passes through the lane 34L, a portion of the hydrogel prepolymer 60-4 on the lane 34L may be is crosslinked via the hydrogel-crosslinking light, while other portions of the hydrogel prepolymer 60-4 that are not crosslinked may be removed (e.g., washed out).

Referring to FIG. 6N, in some embodiments, a hydrogel prepolymer 60-5 is formed (dropped) on the channel waveguide 30′ and at least in contact with the lane 35L. Then, a light source S1 emits a hydrogel-crosslinking light (not labeled in FIG. 6N) having a wavelength of λ1 to the grating coupler 35, and the hydrogel-crosslinking light passes through the lane 35L. Referring to FIG. 6O, in some embodiments, a (crosslinked) hydrogel 65 is formed on the lane 35L to form the biochip 102. Since the hydrogel-crosslinking light merely passes through the lane 35L, a portion of the hydrogel prepolymer 60-5 on the lane 35L may be is crosslinked via the hydrogel-crosslinking light, while other portions of the hydrogel prepolymer 60-5 that are not crosslinked may be removed (e.g., washed out).

As shown in FIG. 6F to 6O, in some embodiments, the hydrogel (e.g., 61, 62, 63, 64, and 65) is disposed on all of the lanes (e.g., 31L, 32L, 33L, 34L, and 35L), and the hydrogel on different lanes has different functional molecules or concentrations. In more detail, the hydrogel prepolymer 60-1, 60-2, 60-3, 60-4 and 60-5 may have different functional molecules or concentrations. Therefore, in this embodiment, the hydrogels 61, 62, 63, 64, and 65 are respectively disposed on the lanes 31L, 32L, 33L, 34L, and 35L, and the hydrogels 61, 62, 63, 64, and 65 have different functional molecules or concentrations.

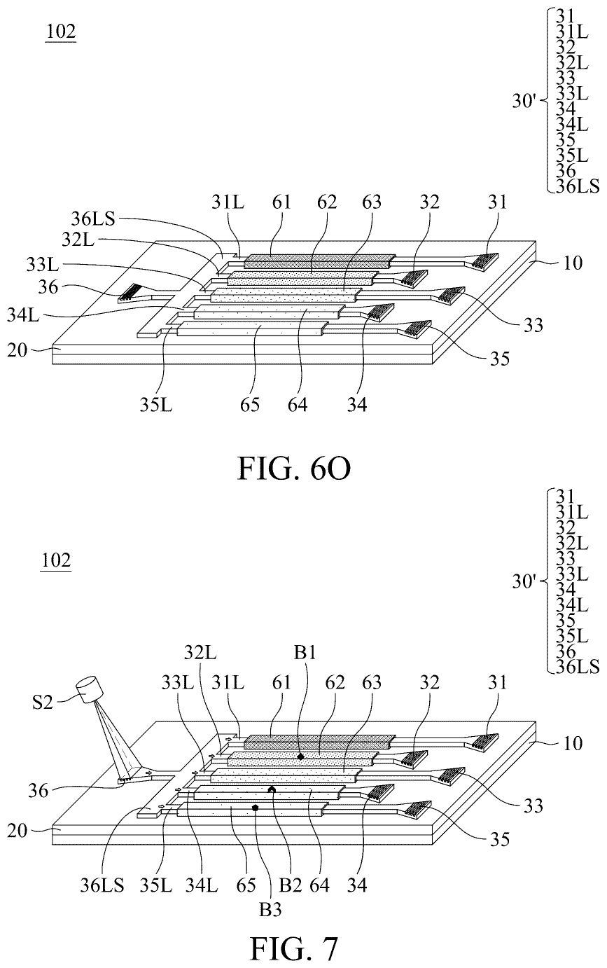

FIG. 7 is a three-dimensional schematic diagram illustrating the biochip 102 according to some embodiments of the present disclosure. It should be noted that some components of the biochip 102 have been omitted in FIG. 7 for the sake of brevity. As shown in FIG. 7, in some embodiments, a light source S2 emits light to the grating coupler 36 of the channel waveguide 30′, and a sensing light having a wavelength of λ2 is split by the light-splitting component 36LS and passes through the lanes 31L, 32L, 33L, 34L, and 35L. In this embodiment, the grating couplers 31, 32, 33, 34, and 35 are used for coupling light having the same or shorter wavelength than the light coupled by the grating coupler 36.

The hydrogels 61, 62, 63, 64, and 65 that include different functional molecules (or different concentrations) may bind analytes with fluorescent tags at lanes 31L, 32L, 33L, 34L, and 35L. After loading biosamples and washing, the specific analytes with fluorescent tags may be immobilized at the hydrogel. Then, by coupling the sensing light that has a wavelength of λ2 into the channel waveguide 30′, the sensing light may propagate in the channel waveguide 30′ and be split into each lane to excite fluorescence molecules and emit fluorescent signals. If the specific analytes exist in the biosample (e.g., biosamples B1, B2, and B3), the hydrogel on the corresponding lane (e.g., lanes 32L, 34L, and 35L) will emit the designed fluorescent signals that can be collected by an objective or a photoelectric conversion element (e.g., photodiode).

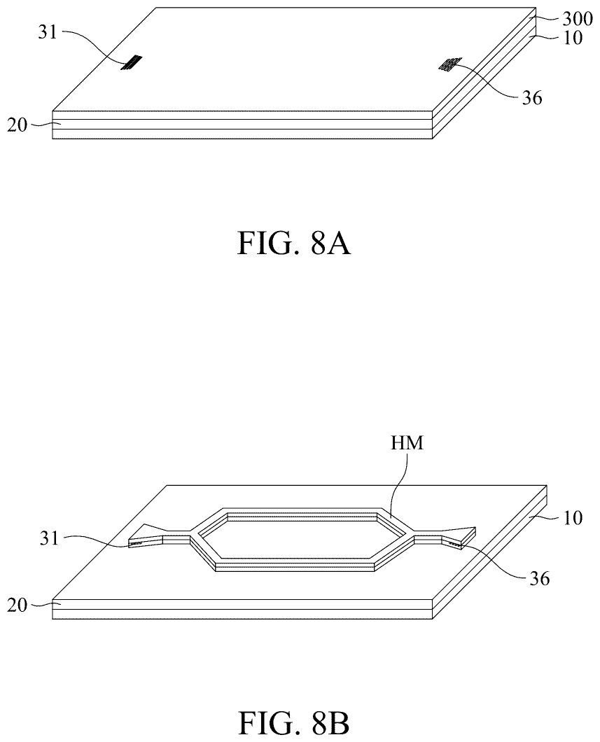

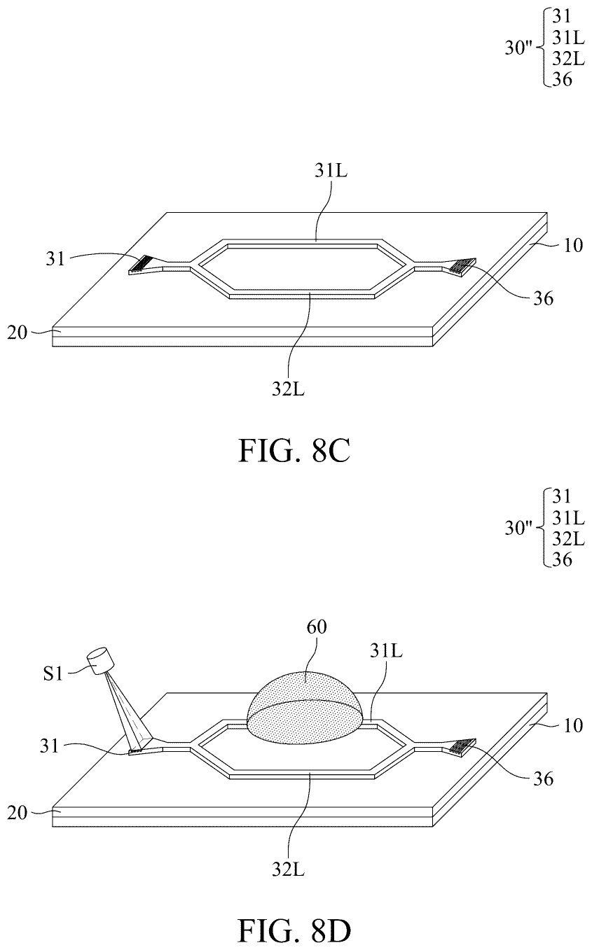

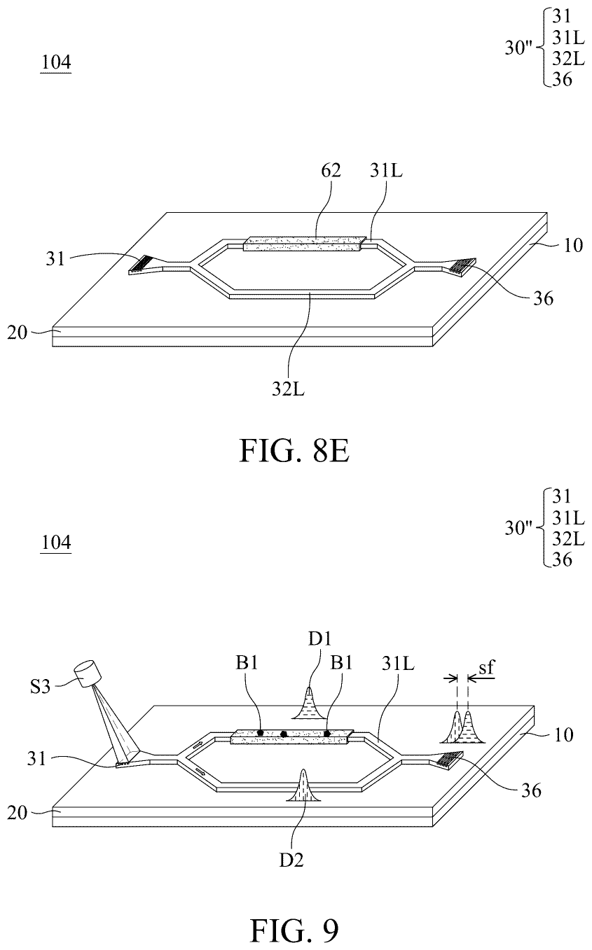

FIG. 8A to FIG. 8E are three-dimensional schematic diagrams illustrating a method for manufacturing the biochip 104 at various stages according to some embodiments of the present disclosure. It should be noted that some components have been omitted in FIG. 8A to FIG. 8E for the sake of brevity. Moreover, FIG. 8A may follow the stage shown in FIG. 6B, but the present disclosure is not limited thereto.

Referring to FIG. 8A, in some embodiments, a grating coupler 31 is formed on one side of the waveguide material layer 300 (which are disposed over left side of the substrate 10), while a grating coupler 36 is formed on another side of the waveguide material layer 300 (which is disposed over right side of the substrate 10). The grating couplers 31 and the grating coupler 36 may be formed by a photolithography process and/or an etching process.

Referring to FIG. 8B and FIG. 8C, in some embodiments, a waveguide material layer 300 is patterned to form a channel waveguide 30″. In more detail, a mask layer HM may be formed on the waveguide material layer 300, then a portion of the waveguide material layer 300 not covered by the mask layer HM is removed to form the channel waveguide 30″. In other words, the waveguide material layer 300 not covered by the mask layer HM may be etched, thereby retaining the channel waveguide 30″.

As shown in FIG. 8C, in some embodiments, the channel waveguide 30″ includes the grating coupler 31 disposed over one side (e.g., left side in FIG. 8C) of the substrate 10, the grating coupler 36 disposed over another side (e.g., right side in FIG. 8C) of the substrate 10, and multiple lanes 31L, 32L that connect the grating coupler 31 to the grating coupler 36.

Referring to FIG. 8D, in some embodiments, a hydrogel prepolymer 60 is formed (dropped) on the lane 31L. Then, a light source S1 emits a hydrogel-crosslinking light (not labeled in FIG. 8D) having a wavelength of λ1 into the grating coupler 31, and the hydrogel-crosslinking light passes through the lane 31L. Referring to FIG. 8E, in some embodiments, a (crosslinked) hydrogel 61 is formed on the lane 31L. Since the hydrogel prepolymer 60 is formed on the lane 31L but not on the lane 32L, a portion of the hydrogel prepolymer 60 on the lane 31L may be is crosslinked via the hydrogel-crosslinking light, while other portions of the hydrogel prepolymer 60 that are not crosslinked may be removed (e.g., washed out).

Therefore, as shown in FIG. 8E, in some embodiments, the hydrogel 62 is disposed on one lane 31L to form a sensing arm, while the other lane 32L that is free of the hydrogel forms a reference arm.

FIG. 9 is a three-dimensional schematic diagram illustrating the biochip 104 according to some embodiments of the present disclosure. It should be noted that some components of the biochip 104 have been omitted in FIG. 9 for the sake of brevity. As shown in FIG. 9, in some embodiments, a light source S3 emits light to the grating coupler 31 of the channel waveguide 30″, and a sensing light having a wavelength of λ3 passes through the lanes 31L and 32L.

After loading biosamples and washing, the specific analytes may be immobilized at the hydrogel. Then, by coupling the sensing light that has a wavelength of λ3 into the channel waveguide 30″, the sensing light may propagate in the channel waveguide 30′ and be split into each lane. If the specific analytes exist in the biosample, the hydrogel 62 on the lane 31L will capture the analytes and increase the refractive index that cause wavelength shift sf (by comparing distribution figure D1 with distribution figure D2). By using the 3D hydrogel 62 with functional molecule, the binding number of the analytes can be increased several times compared to binding number of the analytes on the 2D surface. Therefore, the refractive index change can be increased for better sensitivity.

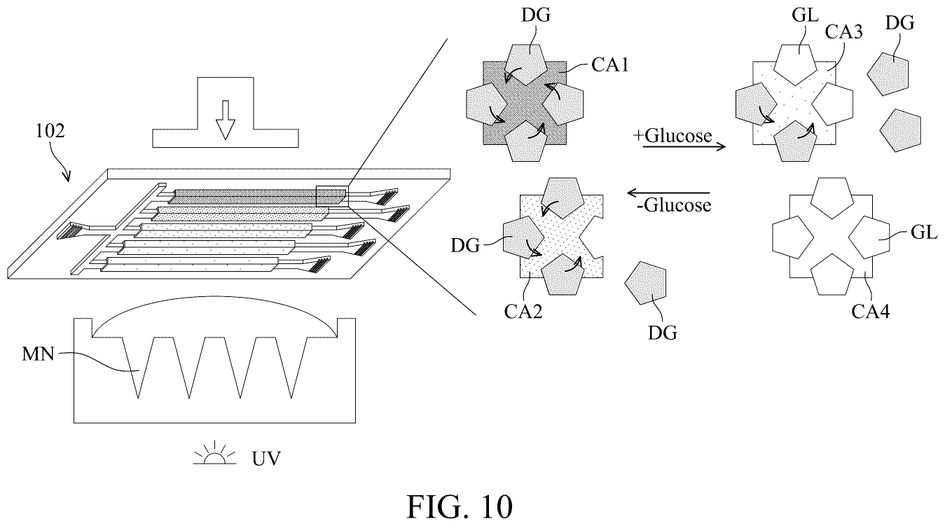

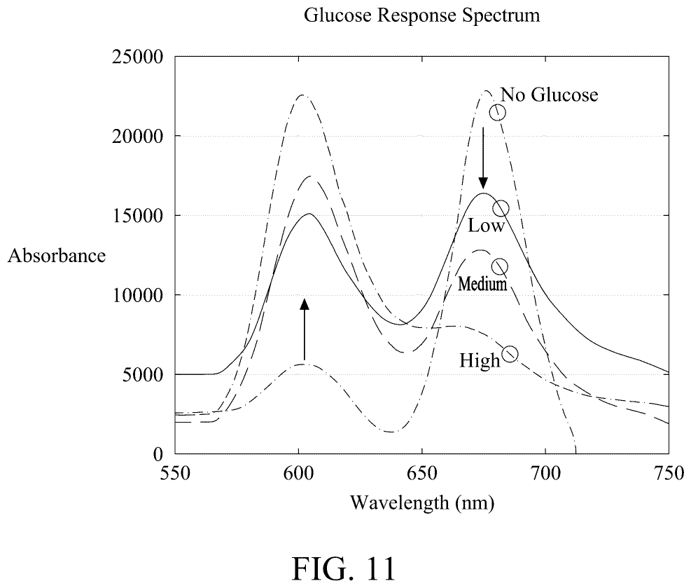

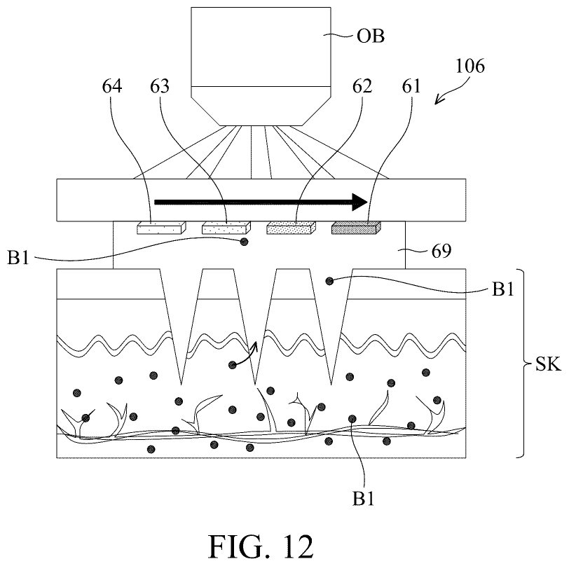

FIG. 10 shows a schematic diagram of the effect of glucose on functional molecules in hydrogel 62 of the biochip 102. FIG. 11 is a glucose response spectrum. FIG. 12 shows an application of the biochip 106 on the skin SK of living organisms according to some embodiments of the present disclosure.

As shown in FIG. 10, the biochip 102 may form multiple hydrogel line arrays, with the hydrogel (e.g., hydrogels 61, 62, 63, 64, and 65) on different lanes (e.g., lanes 31L, 32L, 33L, 34L, and 35L) containing different functional molecules or concentrations. Therefore, the biochip 102 may be used to detect the concentration gradient of a target biosample or the presence of various specific substances in the target biosample. One possible implementation is to replicate the microneedle structure (by using a microneedle mold, injecting hydrogel prepolymer, then covering the microneedle mold with the biochip 102 shown in FIG. 7, and curing the microneedle mold by exposing hydrogel crosslinking light UV below the microneedle mold to form the microneedle MN), integrating the microneedle fabrication onto the biochip 102. Concanavalin A-dextran FRET complexes with different concentrations may be embedded in the hydrogels 61, 62, 63, 64, and 65. The concanavalin A (labeled as CA1 in FIG. 10) is marked with Alexa 647 (ex/em 650/671 nm), and dextran (labeled as DG in FIG. 10) is marked with Alexa 568 (ex/em 578/603 nm). Since the emission wavelength of Alexa 568 overlaps with the excitation wavelength of Alexa 647, when concanavalin A-dextran forms a complex, the Alexa 568 and Alexa 647 marked thereon are very close (<10 nm). In this case, when the excitation light excites Alexa 568, part of the light emitted by Alexa 568 is absorbed by Alexa 647, which then emits light at the Alexa 647 emission wavelength. Therefore, the concanavalin A-dextran FRET complex, as shown in FIG. 11, in the absence of external glucose, shows that when excited with green light, the complex of concanavalin A-Alexa 647 and dextran-Alexa 568 will emit light around 600 nm, most of which will be absorbed by Alexa 647 on concanavalin A, resulting in a high fluorescence intensity at 670 nm and a low fluorescence intensity at 600 nm. When glucose is present, glucose, which has a higher affinity for concanavalin A, will replace dextran-Alexa 568, causing dextran-Alexa 568 to move away from concanavalin A, and the emission from dextran's Alexa 568 will no longer be absorbed by concanavalin A. The more glucose replaces the position of dextran, the more light emitted by dextran-Alexa 568 will not be absorbed by Alexa 647 on concanavalin A. Therefore, as shown in FIG. 11, by observing the change in the intensity ratio between the 670 nm and 600 nm emission peaks, the amount of glucose in the interstitial fluidinsulin sensitivity factor (ISF) may be inferred, which in turn indicates the blood glucose concentration within the skin. Additionally, by using hydrogel line arrays with different initial concentrations of the concanavalin A-dextran FRET complex, it is possible to perform multi-point detection on a single glucose sample, thereby increasing the accuracy of the measurement.

When the microneedle MN pierces the skin, it absorbs the interstitial fluidinsulin sensitivity factor (ISF) (e.g., glucose, lactate, or alcohol) and diffuses these substances into the hydrogels 61-65. Since the amount of ISF that can be absorbed after microneedle insertion is generally a few microliters, the glucose within will replace the position of DG in the original DG+CA1 or DG+CA2 complex, resulting in a different 660/670 nm intensity ratio when excited by green light, which may then be used to determine the amount and concentration of glucose in the test sample.

As shown in FIG. 12, the biochip 106 may have a similar structure to the biochip 102 shown in FIG. 7. In some embodiments, the biochip 106 further includes a microneedle structure 69 that connects the hydrogels 61, 62, 63, and 64 to an external component (e.g., the skin SK of living organisms). The microneedle structure 69 that integrates on a biosensor (which includes biochip 106) may help to penetrate skin and extract interstitial fluidinsulin sensitivity factor (e.g., glucose, lactate, or alcohol) for physiological signal monitoring.

As noted above, the biochip according to the embodiments of the present disclosure includes a waveguide core layer. The waveguide core layer includes at least one grating coupler for coupling light to crosslink the hydrogel, so as to improve selective modification and/or multiplex screening capabilities of the biochip via thin hydrogel.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure. Therefore, the scope of protection should be determined through the claims. In addition, although some embodiments of the present disclosure are disclosed above, they are not intended to limit the scope of the present disclosure.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the disclosure.

What is claimed is:

1. A biochip, comprising:
a substrate;
a waveguide core layer disposed over the substrate, wherein the waveguide core layer comprises a first grating coupler; and
a hydrogel crosslinked via a hydrogel-crosslinking light that is coupled by the first grating coupler.

2. The biochip as claimed in claim 1, wherein the substrate has a photoelectric conversion element, and the hydrogel corresponds to the photoelectric conversion element.

3. The biochip as claimed in claim 2, further comprising:
an upper cladding layer disposed on the waveguide core layer and comprising a nano-well that is disposed over the photoelectric conversion element, wherein the upper cladding layer exposes the first grating coupler, and the hydrogel is disposed at the bottom of the nano-well.

4. The biochip as claimed in claim 3, wherein a thickness of the upper cladding layer is greater than 50 nm.

5. The biochip as claimed in claim 4, wherein when the thickness of the upper cladding layer is greater than 100 nm, a distance between the topmost of the hydrogel and the waveguide core layer is less than 100 nm.

6. The biochip as claimed in claim 3, wherein the substrate has multiple photoelectric conversion elements and the upper cladding layer comprises multiple nano-wells that are disposed over the photoelectric conversion elements, and there are multiple hydrogels disposed at the bottoms of the nano-wells.

7. The biochip as claimed in claim 3, further comprising:
a self-assembled monolayer disposed on the upper cladding layer and between the upper cladding layer and the hydrogel.

8. The biochip as claimed in claim 1, wherein the first grating coupler is used for coupling a hydrogel-crosslinking light and a sensing light, and a wavelength of the hydrogel-crosslinking light is shorter than a wavelength of the sensing light.

9. The biochip as claimed in claim 1, wherein the first grating coupler is used for coupling a hydrogel-crosslinking light and the waveguide core layer further comprises:
a second grating coupler on the opposite side of the first grating coupler, wherein the second grating coupler is used for coupling a sensing light, and a wavelength of the hydrogel-crosslinking light is shorter than a wavelength of the sensing light.

10. The biochip as claimed in claim 1, further comprising:
a lower cladding layer disposed between the substrate and the waveguide core layer.

11. The biochip as claimed in claim 1, wherein the waveguide core layer is formed as a channel waveguide, and the channel waveguide comprises:
at least one first grating coupler disposed over one side of the substrate;
a second grating coupler disposed over another side of the substrate; and
multiple lanes connecting the at least one first grating coupler to the second grating coupler.

12. The biochip as claimed in claim 10, wherein there are multiple interleaved first grating couplers.

13. The biochip as claimed in claim 12, wherein the channel waveguide further comprises:
a light-splitting component connecting the second grating coupler to the lanes.

14. The biochip as claimed in claim 12, wherein the first grating couplers are arranged in an array.

15. The biochip as claimed in claim 11, wherein the hydrogel is disposed on all of the lanes, and the hydrogel on different lanes has different functional molecules or concentrations.

16. The biochip as claimed in claim 11, wherein the at least one first grating coupler is used for coupling light having the same or shorter wavelength than a light coupled by the second grating coupler.

17. The biochip as claimed in claim 11, wherein the hydrogel is disposed on one of the lanes to form a sensing arm, while the other of the lanes that is free of the hydrogel forms a reference arm.

18. The biochip as claimed in claim 1, wherein the hydrogel comprises gelatin methacrylate, polyethylene glycol diacrylate, or hyaluronic acid.

19. The biochip as claimed in claim 1, wherein the hydrogel comprises multiple functional molecules, and the functional molecules comprises DNA primer, concanavalin A-dextran FRET complex, or antibodies.

20. The biochip as claimed in claim 1, further comprising:
a microneedle structure connecting the hydrogel to an external component.
